SEQUENCE LISTING (6 sequences)

SEQ ID NO: 1 - protein, 487 aa, bacteriophage T4
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 2 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIKELTGYITDGKKDDVEVLRRVMMRDLECGASRSIANKVWPKLIPEQPQMLASSYDEKGINKNIKFPAFAQLKADGARCFAKVRGDELDDVRLLSRAGNEYLGLDLLKEELIKMTAEARQIHPEGVLIDGELVYHEKVKKEPEGADFLFDAYPENSKAKEFAEVAESRTASNGIANKSLKGTISEKEAQCMKFQVWDYVPLVEIYSEPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECKCNGWLKSDGRTDYVKLFLPIAIRLREDKTKANTFEDVFGDFHEVTGL

SEQ ID NO: 3 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIKELTGYITDGKKDDVEVLRRVMMRDLECGASRSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEKVKKEPEGLDFLFDAYPENSKAKEFAEVAESRTASNGIANKSLKGTISEKEAQCMKFQVWDYVPLVEIYSEPAFRLKYDVRFSKLEQMTSGYDKVILIENQVVNNLDEAKVIYKKYIDQGLEGIILKNIDGLWENARSKNLYKFKEVIDIDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHKVTGL

SEQ ID NO: 4 - DNA, 1461 bp, bacteriophage T4
  source 1..1461: mol_type = genomic DNA
atgattcttaagattctgaatgaaatcgcgagcatcggtagcacgaagcagaaacaggccatccttgaaaaaaataaagacaacgaactcttgaagcgcgtctaccgtcttacctacagccgcggtctgcagtactatattaaaaaatggccaaaaccgggaatcgcaactcaatcgttcggcatgcttaccctcaccgatatgctggactttatcgagtttaccttggctacccgcaaactgactggcaatgccgctatcgaagaactgactggatacattacggatggtaaaaaagatgatgtcgaagtgttgcggcgtgtcatgatgcgcgatctggagtgtggcgcatcggtgagcatcgcaaacaaggtttggcctggattaattccggaacagccacagatgcttgcttcatcttacgatgagaaaggcattaacaaaaatatcaaatttccagcctttgcgcaactgaaagcagacggcgctcgttgtttcgcggaagtccgcggtgatgaattggatgatgtccgtctgctcagccgcgcagggaacgaatatctgggcttggacttgctgaaagaggaactgattaagatgaccgccgaagcacgccagatccacccagaaggcgtcctgattgatggtgaattggtctaccatgaacaggtgaaaaaagaaccggaaggcttagattttctctttgacgcatatccggaaaactcgaaagctaaagaattcgctgaagttgccgaaagccggacggcgagcaacggtatcgcgaacaaaagcttgaaaggcacgatcagcgagaaggaggcacagtgtatgaagtttcaggtgtgggattatgttccgcttgtagagatttattcgttacccgccttccgtttaaagtacgatgttcgctttagcaaacttgaacagatgacgagcggatatgacaaagtgattctcatcgaaaatcaggtggtcaacaatctggacgaggccaaagtgatctataaaaaatatatcgaccaggggcttgaaggtattattttgaaaaacatcgacggcttgtgggaaaacgcgcggagcaaaaatctgtataaatttaaagaggttatcgacgtggacttaaagatcgttggaatttacccgcatcggaaagacccgacgaaagcgggtggctttatcctggagtctgaatgcggcaaaattaaagtgaatgctggctctggactgaaagacaaagccggtgtaaaaagtcatgagctggatcgtactcgcattatggaaaatcagaattattatattggtaaaatcttggaatgtgagtgcaacggctggctgaaatcggacgggcgtaccgattacgtgaaactgttcttgccgattgctattcggttacgcgaagacaaaactaaagctaataccttcgaagatgtgttcggcgatttccacgaggtgaccggcctt

SEQ ID NO: 5 - DNA, 1461 bp, synthetic construct
  source 1..1461: mol_type = other DNA
atgattcttaagattctgaatgaaatcgcgagcatcggtagcacgaagcagaaacaggccatccttgaaaaaaataaagacaacgaactcttgaagcgcgtctaccgtcttacctacagccgcggtctgcagtactatattaaaaaatggccaaaaccgggaatcgcaactcaatcgttcggcatgcttaccctcaccgatatgctggactttatcgagtttaccttggctacccgcaaactgactggcaatgccgctatcaaagaactgactggatacattacggatggtaaaaaagatgatgtcgaagtgttgcggcgtgtcatgatgcgcgatctggagtgtggcgcatcgcgtagcatcgcaaacaaggtttggcctaaattaattccggaacagccacagatgcttgcttcatcttacgatgagaaaggcattaacaaaaatatcaaatttccagcctttgcgcaactgaaagcagacggcgctcgttgtttcgcgaaagtccgcggtgatgaattggatgatgtccgtctgctcagccgcgcagggaacgaatatctgggcttggacttgctgaaagaggaactgattaagatgaccgccgaagcacgccagatccacccagaaggcgtcctgattgatggtgaattggtctaccatgaaaaagtgaaaaaagaaccggaaggcgcagattttctctttgacgcatatccggaaaactcgaaagctaaagaattcgctgaagttgccgaaagccggacggcgagcaacggtatcgcgaacaaaagcttgaaaggcacgatcagcgagaaggaggcacagtgtatgaagtttcaggtgtgggattatgttccgcttgtagagatttattcggaacccgccttccgtttaaagtacgatgttcgctttagcaaacttgaacagatgacgagcggatatgacaaagtgattctcatcgaaaatcaggtggtcaacaatctggacgaggccaaagtgatctataaaaaatatatcgaccaggggcttgaaggtattattttgaaaaacatcgacggcttgtgggaaaacgcgcggagcaaaaatctgtataaatttaaagaggttatcgacgtggacttaaagatcgttggaatttacccgcatcggaaagacccgacgaaagcgggtggctttatcctggagtctgaatgcggcaaaattaaagtgaatgctggctctggactgaaagacaaagccggtgtaaaaagtcatgagctggatcgtactcgcattatggaaaatcagaattattatattggtaaaatcttggaatgtaaatgcaacggctggctgaaatcggacgggcgtaccgattacgtgaaactgttcttgccgattgctattcggttacgcgaagacaaaactaaagctaataccttcgaagatgtgttcggcgatttccacgaggtgaccggcctt

SEQ ID NO: 6 - DNA, 1461 bp, synthetic construct; identical to SEQ ID NO: 5
  source 1..1461: mol_type = other DNA

(19) United States
(12) Patent Application Publication
(10) Pub. No.: US 2026/0201359 A1
(43) Pub. Date: Jul. 16, 2026

(54) ENZYMES FOR LIBRARY PREPARATION

(71) Applicant: Twist Bioscience Corporation, South San Francisco, CA (US)

(72) Inventors: Sean Patrick TIGHE, Soquel, CA (US); Owen Kabnick SMITH, Mountain View, CA (US); Elian LEE, San Mateo, CA (US); Ramsey Ibrahim ZEITOUN, Westminster, CO (US); Siyuan CHEN, Palo Alto, CA (US); Adil YUSUF, Lexington, KY (US); Mohamed Hassan KANE, Chelsea, MA (US)

(73) Assignee: Twist Bioscience Corporation, South San Francisco, CA (US)

(21) Appl. No.: 19/136,082

(22) PCT Filed: Dec. 5, 2023
(86) PCT No.: PCT/US2023/082433

§ 371 Date: Jun. 5, 2025

Related U.S. Application Data
(60) Provisional application No. 63/386,143, filed on Dec. 5, 2022.

(51) Int. Cl. C12N 9/00

(52) U.S. Cl. CPC C12N 9/93

(57) ABSTRACT

Provided herein are methods and compositions relating to enzymes and libraries having nucleic acids encoding for an enzyme comprising modified sequences. Further provided herein are methods for enzyme optimization. Further provided herein are enzymes for sequencing library generation.

### CROSS-REFERENCE TO RELATED APPLICATION

This application claims priority to U.S. Provisional Patent Application No. 63/386,143, filed on Dec. 5, 2022, which is hereby incorporated by reference in its entirety. All publications, patents, and patent applications mentioned in this specification are herein incorporated by reference to the same extent as if each individual publication, patent, or patent application was specifically and individually indicated to be incorporated by reference.

### BACKGROUND

Enzymes possess the capability to catalyze a wide range of chemical reactions, including those used in chemical biology for sequencing applications. However, the design and implementation of enzymes can be challenging. Thus, there is a need to develop compositions and methods for the optimization of enzyme properties.

### BRIEF SUMMARY

Provided herein are methods, compositions, and systems for the optimization of enzymes.

Provided herein are variant polypeptides comprising at least one amino acid mutation relative to SEQ ID NO.: 1. Further provided herein are variant polypeptides wherein the polypeptide comprises at least 80% similarity to any one of SEQ ID NOS: 2-3. Further provided herein are variant polypeptides wherein the polypeptide comprises at least 90% similarity to any one of SEQ ID NOS: 2-3. Further provided herein are variant polypeptides wherein the polypeptide comprises at least 95% similarity to any one of SEQ ID NOS: 2-3. Further provided herein are variant polypeptides wherein the polypeptide comprises at least 98% similarity to any one of SEQ ID NOS: 2-3. Further provided herein are variant polypeptides wherein the polypeptide comprises any one of SEQ ID NOS: 2-3. Further provided herein are variant polypeptides wherein the polypeptide comprises at least 10 contiguous amino acids of any one of SEQ ID NOS: 2-3. Further provided herein are variant polypeptides wherein the polypeptide comprises at least 20 contiguous amino acids of any one of SEQ ID NOS: 2-3. Further provided herein are variant polypeptides wherein the polypeptide comprises 20-100 contiguous amino acids of any one of SEQ ID NOS: 2-3. Further provided herein are variant polypeptides wherein the polypeptide comprises at least 2 amino acid mutations relative to SEQ ID NO: 1. Further provided herein are variant polypeptides wherein the polypeptide comprises at least 4 amino acid mutations relative to SEQ ID NO: 1. Further provided herein are variant polypeptides wherein the polypeptide comprises at least 6 amino acid mutations relative to SEQ ID NO: 1. Further provided herein are variant polypeptides wherein the mutations are at one or more of positions E88, T91, V119, G128, E168, Q223, L231, L293, V372, E440, D448, and E483 relative to SEQ ID NO.: 1. Further provided herein are variant polypeptides wherein the mutations are at one or more of positions E88, V119, G128, E168, Q223, L231, L293, and E440 relative to SEQ ID NO.: 1. Further provided herein are variant polypeptides wherein mutations are at one or more of positions E88, V119, Q223, L293, V372, and E483 relative to SEQ ID NO.: 1. Further provided herein are variant polypeptides wherein the mutations are selected from one or more of E88K, T91M, V119R, G128K, E168K, Q223K, L231A, L293E, V372I, E440K, D448W, D448P, and E483K relative to SEQ ID NO.: 1. Further provided herein are variant polypeptides wherein the mutations are selected from one or more of E88K, V119R, G128K, E168K, Q223K, L231A, L293E, and E440K relative to SEQ ID NO.: 1. Further provided herein are variant polypeptides wherein the mutations are selected from one or more of E88K, V119R, Q223K, L293E, V372I, and E483K relative to SEQ ID NO.: 1. Further provided herein are variant polypeptides wherein the polypeptide further comprises a purification tag.

Provided herein are nucleic acids encoding for a polypeptide of described herein. Further provided herein are nucleic acid comprising at least 80% similarity to any one of SEQ ID NOS: 4-5, with the proviso the polypeptide does encode for a polypeptide of SEQ ID NO.: 1. Further provided herein are nucleic acids wherein the nucleic acid of comprises at least 90% similarity to any one of SEQ ID NOS: 4-5. Further provided herein are nucleic acids wherein the nucleic acid of comprises at least 95% similarity to any one of SEQ ID NOS: 4-5.

Provided herein are vector comprising the nucleic acid described herein. Further provided herein are vectors wherein the vector comprises a plasmid. Provided herein are cells comprising the nucleic acids described herein. Further provided herein are cells wherein the cell comprises a bacterial cell.

Provided herein are methods of expressing a polypeptide disclosed herein. Further provided herein are methods wherein expression comprises translation of the nucleic acid sequences provided herein. Further provided herein are methods wherein the method comprises an in-vivo method. Further provided herein are methods wherein the method comprises a cell-free method.

Provided herein are methods for forming a covalent bond between two nucleotides comprising contacting a first nucleotide and a second nucleotide with a polypeptide disclosed herein. Further provided herein are methods wherein the first nucleotide and the second nucleotide are present on the same nucleic acid. Further provided herein are methods wherein the covalent bond forms a circular nucleic acid. Further provided herein are methods wherein the first nucleotide is present on a first nucleic acid and the second nucleotide is present on a second nucleic acid. Further provided herein are methods wherein the first nucleic acid and/or the second nucleic comprises genomic DNA or a fragment thereof. Further provided herein are methods wherein the first nucleic acid and/or the second nucleic comprises cDNA. Further provided herein are methods wherein the first nucleic acid and/or the second nucleic comprises an adapter. Further provided herein are methods wherein the first nucleic acid comprises a first adapter and genomic DNA or cDNA. Further provided herein are methods wherein the second nucleic acid comprises a second adapter. Further provided herein are methods wherein the adapter comprises at least one barcode. Further provided herein are methods wherein the barcode comprises one or more of a sample index, a plate index, a cell index, and a unique molecular identifier.

Provided herein are methods wherein for preparing a nucleic acid library comprising (a) providing one or more sample nucleic acids; (b) contacting the one or more sample nucleic acids with a plurality of adapters and a polypeptide disclosed herein to form a nucleic acid sequencing library comprising adapter-ligated nucleic acids; and (c) sequencing the nucleic acid library. Further provided herein are methods wherein the sample nucleic acids comprise genomic fragments. Further provided herein are methods wherein the genomic fragments are obtained from cleavage or amplification of a genome. Further provided herein are methods wherein the sample nucleic acids comprise cDNAs. Further provided herein are methods wherein the sample nucleic acids comprise cfDNAs. Further provided herein are methods wherein the method further comprises one or more steps of end-repair, a-tailing, and amplification. Further provided herein are methods wherein the method further comprises enriching the nucleic acid library prior to sequencing.

### BRIEF DESCRIPTION OF THE DRAWINGS

FIG. 1 depicts an automated workflow for optimizing ligation enzymes.

FIGS. 2A-2B depict a strategy for designing ligase variants with MSA from high entropy positions. FIG. 2A depicts a plot of cumulative probabilities for amino acids (0.0 to 1.0 at 0.2 unit intervals) vs. Position in T4 ligase (left to right: 212-214, 222-224, 272-274, 296-298, 308-310). FIG. 2B depicts a plot of Shannon entropy (0.0 to 3.0 at 0.5 unit intervals) vs. Position in T4 ligase (left to right: 212-214, 222-224, 272-274, 296-298, 308-310).

FIG. 3A depicts a workflow for high throughput cell free screening of T4 Ligase and SYBR green qPCR for quantification.

FIG. 3B depicts an amplification plot obtained from the workflow in FIG. 3A. The y-axis is labeled RFUs (fluorescence units from 0 to 4000, 1000 unit intervals); the x-axis is labeled PCR cycles (from 0 to 40 at 10 unit intervals).

FIG. 4 depicts plots obtained from a first round single variant screen. Left to right: activity, thermostability, and salt.

FIG. 5A depicts a heat map from a first round screen of single variants. Red indicates higher activity, blue indicates lower activity. The legend shows colors corresponding to activity from −2 to 3 at 1 unit intervals.

FIG. 5B depicts a heat map from a second round screen using binary combinations of single variants to measure epistatic effects. Blue indicates higher activity, red indicates lower activity. The legend shows colors corresponding to activity (units in ct values) from −4 to 6 at 1 unit intervals.

FIG. 6A depicts a plot obtained from rounds 4/5 using raw addition of single variants. The x-axis is labeled Activity from 10 to 18 at 1 unit intervals; the y-axis is labeled proportion from 0.0 to 1.0 at 0.2 unit intervals.

FIG. 6B depicts a plot obtained from rounds 4/5 using raw addition of single variants. The x-axis is labeled number of variants (left to right: 3, 4, 5, 6); the y-axis is labeled Activity (1/(2\hat{ }ct)) from 0.0000 to 0.0014 at 0.0002 unit intervals.

FIG. 7 depicts an SDS-PAGE gel used to prepare molecular biology grade ligase from variants. Lanes: (1) ladder; (2) lysate; (3) flow through; (4) blank; (5-10) ligases.

FIG. 8A depicts structural information on T4 ligase variant designs. Numerous lysine mutations (+charge) were observed near DNA substrate for variants.

FIG. 8B depicts structural information on T4 ligase variant designs. Residues contacting the DNA substrate are shown with boxes and include positions 14, 15, 16, 39, 44, 46, 48, 49, 79, 82, 84, 116, 118, 119, 120, 121, 124, 157, 159, 164, 181, 182, 185, 217, 254, 258, 262, 263, 266, 268, 282, 361, 380, 382, 383, 384, 404, 406, 407, 410, 411, 412, 447, 448, 450, 455, 457, 458, 459, and 460.

FIG. 9A depicts percent chimera for a series of variant T4 ligases. The y-axis is labeled Percent_Chimera from 0.000 to 0.030 at 0.005 unit intervals. Variants from various rounds of selection are labeled on the x-axis: 38, 6_1, 6_16, 6_8, 7_1, 7_10, 7_11, 7_12, 7_13, 7_14, 7_15, 7_16, 7_18, 7_19, 7_2, 7_20, 7_3, 7_6, 7_8, 7_9, AZ, E12, Qiagen, and WT.

FIG. 9B depicts an example of a chimera formed from two biological sequences.

FIG. 10A depicts a 2D plot of chimera vs. activity. The y-axis is labeled chimera from 21 to 29 at 1 unit intervals. The x-axis is labeled activity from 10.0 to 30.0 at 2.5 unit intervals. The legend is labeled data (blue), ngs samples (orange), green (low chimera), red (seq38), wt (purple), and singles (brown).

FIG. 10B depicts a 2D plot of chimera vs. activity. The y-axis is labeled chimera from 21 to 26 at 1 unit intervals. The x-axis is labeled activity from 12 to 20 at 1 unit intervals. The heatmap legend is labeled adapters only CT from 8 (dark blue) to 18 (light blue) at 2 unit intervals. Seq38 is indicated.

FIG. 10C depicts a single site mutagenesis library for a portion of the T4 ligase sequence.

FIG. 11A depicts a plot of variant performance relative to sequence 38, over four NGS runs. The y-axis is labeled Variant/38 total reads from 0.0 to 1.0 at 0.2 unit intervals. The x-axis is labeled with variants (left to right): r7r-18, r7r-24, r7r-12, r7r-8, r7r-21, 6-8, r7r-22, r7r-2, r7r-34, 6-1, r7r-5, r7r-1, 7-19, r7r-15, r7r-25, r7r-13, r7r-19, r7r-20, r7r-32, r7r-4, r7r-29, r7r-6, r7r-17, r7r-26, r7r-30, r7r-3, r7r-23, r7r-9, r7r-33, r7r-11, 7-28, r7r-14, r7r-7, r7r-10, r7r-16, r7r-31, r7r-28, r7r-27, wt).

FIG. 11B depicts a plot of variant chimera relative to sequence 38, over four NGS runs. The y-axis is labeled Variant/38% chimera from 0.0 to 1.0 at 0.2 unit intervals. The x-axis is labeled with variants IDs (left to right): r7r-18, r7r-24, r7r-12, r7r-8, r7r-21, 6-8, r7r-22, r7r-2, r7r-34, 6-1, r7r-5, r7r-1, 7-19, r7r-15, r7r-25, r7r-13, r7r-19, r7r-20, r7r-32, r7r-4, r7r-29, r7r-6, r7r-17, r7r-26, r7r-30, r7r-3, r7r-23, r7r-9, r7r-33, r7r-11, 7-28, r7r-14, r7r-7, r7r-10, r7r-16, r7r-31, r7r-28, r7r-27, wt).

FIG. 12A depicts a plot of total reads for all titrations of enzyme amount in a ligase experiment. The y-axis is labeled total reads from 0 to 800,000 at 200,000 unit intervals. The x-axis is labeled with variants and amounts (in ng) (left to right): 12-1000, 12-500, 12-250, 18-1000, 18-500, 18-250, 21-1000, 21-500, 21-500, 22-1000, 22-500, 22-250, 24-1000, 24-500, 24-250, 38-1000, 38-500, 38-250, 8-1000, 8-500, 8-250, wt-1000, wt-500, wt-250).

FIG. 12B depicts a plot of percent chimera for all titrations of enzyme amount in a ligase experiment. The y-axis is labeled total reads from 0.000 to 0.016 at 0.002 unit intervals. The x-axis is labeled with variants and amounts (in ng) (left to right): 12-1000, 12-500, 12-250, 18-1000, 18-500, 18-250, 21-1000, 21-500, 21-500, 22-1000, 22-500, 22-250, 24-1000, 24-500, 24-250, 38-1000, 38-500, 38-250, 8-1000, 8-500, 8-250, wt-1000, wt-500, wt-250).

FIG. 13 depicts a plot of sequencing performance for two variant T4 ligases and wild type (left to right, variant 21, variant 24, and wt). The y-axis is labeled reads converted (normalized) from 0.00 to 2.00 at 0.25 unit intervals. Each set of three bars indicates the enzyme mass/rxn (left to right: 250, 500, 1000 ng).

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence.

### Enzyme Variants

Provided herein are variants of enzymes. In some instances, the enzyme comprises an enzyme for next generation sequencing. In some instances an enzyme comprises a ligase, polymerase, kinase, nuclease, phosphatase, methylase, topoisomerase, transferase, or other enzyme. In some instances, the enzyme comprises a T4 ligase. In some instances, a T4 ligase is selected from Table 1. In some instances, an enzyme comprises a variant of SEQ ID NO. 1.

An enzyme provided herein may comprise one or more variants of SEQ ID NO.: 1. In some instances, a variant comprises at least 1, at least 2, at least 3, at least 4, at least 5, at least 6, at least 7, at least 8, at least 9, at least 10, at least 11, at least 12, at least 13, at least 14, at least 15, or at least 16 variant amino acid positions of SEQ ID NO.: 1. In some instances, a variant comprises about 1, about 2, about 3, about 4, about 5, about 6, about 7, about 8, about 9, about 10, about 11, about 12, about 13, about 14, about 15, or about 16 variant amino acid positions of SEQ ID NO.: 1. In some instances, an enzyme comprises a mutation at one or more of positions selected from 88, 91, 119, 128, 168, 223, 231, 293, 372, 440, 448, or 483 relative to SEQ ID NO.: 1. In some instances, an enzyme comprises a mutation at two or more of positions selected from 88, 91, 119, 128, 168, 223, 231, 293, 372, 440, 448, or 483 relative to SEQ ID NO.: 1. In some instances, an enzyme comprises a mutation at three or more of positions selected from 88, 91, 119, 128, 168, 223, 231, 293, 372, 440, 448, or 483 relative to SEQ ID NO.: 1. In some instances, an enzyme comprises a mutation at four or more of positions selected from 88, 91, 119, 128, 168, 223, 231, 293, 372, 440, 448, or 483 relative to SEQ ID NO.: 1. In some instances, an enzyme comprises a mutation at five or more of positions selected from 88, 91, 119, 128, 168, 223, 231, 293, 372, 440, 448, or 483 relative to SEQ ID NO.: 1. In some instances, an enzyme comprises a mutation at six or more of positions selected from 88, 91, 119, 128, 168, 223, 231, 293, 372, 440, 448, or 483 relative to SEQ ID NO.: 1. In some instances, an enzyme comprises a mutation at seven or more of positions selected from 88, 91, 119, 128, 168, 223, 231, 293, 372, 440, 448, or 483 relative to SEQ ID NO.: 1. In some instances, an enzyme comprises a mutation at eight or more of positions selected from 88, 91, 119, 128, 168, 223, 231, 293, 372, 440, 448, or 483 relative to SEQ ID NO.: 1. In some instances, an enzyme comprises a mutation at nine or more of positions selected from 88, 91, 119, 128, 168, 223, 231, 293, 372, 440, 448, or 483 relative to SEQ ID NO.: 1. In some instances, an enzyme comprises a mutation at ten or more of positions selected from 88, 91, 119, 128, 168, 223, 231, 293, 372, 440, 448, or 483 relative to SEQ ID NO.: 1.

In some instances, an enzyme provided herein comprises the amino acid sequence of any one of SEQ ID NOS.: 2-3. In some instances, an enzyme provided herein comprises the nucleic acid sequence of any one of SEQ ID NOS.: 5-6. Sequences provided herein in some instances comprise a purification tag. In some instances a purification tag comprises a His6 tag.

TABLE 1
Enzyme Protein Sequences

| SEQ ID | | |
|---|---|---|
| NO | Name | Protein Sequence |
| 1 | WT_ | MILKILNEIASIGSTKQKQAILEKNKDNEL |
| | T4_ | LKRVYRLTYSRGLQYYIKKWPKPGIATQSF |
| | Ligase | GMLTLTDMLDFIEFTLATRKLTGNAAIEEL |
| | | TGYITDGKKDDVEVLRRVMMRDLECGASVS |
| | | IANKVWPGLIPEQPQMLASSYDEKGINKNI |
| | | KFPAFAQLKADGARCFAEVRGDELDDVRLL |
| | | SRAGNEYLGLDLLKEELIKMTAEARQIHPE |
| | | GVLIDGELVYHEQVKKEPEGLDFLFDAYPE |
| | | NSKAKEFAEVAESRTASNGIANKSLKGTIS |
| | | EKEAQCMKFQVWDYVPLVEIYSLPAFRLKY |
| | | DVRFSKLEQMTSGYDKVILIENQVVNNLDE |
| | | AKVIYKKYIDQGLEGIILKNIDGLWENARS |
| | | KNLYKFKEVIDVDLKIVGIYPHRKDPTKAG |
| | | GFILESECGKIKVNAGSGLKDKAGVKSHEL |
| | | DRTRIMENQNYYIGKILECECNGWLKSDGR |
| | | TDYVKLFLPIAIRLREDKTKANTFEDVFGD |
| | | FHEVTGL |
| 2 | wt_ | MILKILNEIASIGSTKQKQAILEKNKDNEL |
| | E88K_ | LKRVYRLTYSRGLQYYIKKWPKPGIATQSF |
| | V119R_ | GMLTLTDMLDFIEFTLATRKLTGNAAIKEL |
| | G128K_ | TGYITDGKKDDVEVLRRVMMRDLECGASRS |
| | E168K_ | IANKVWPKLIPEQPQMLASSYDEKGINKNI |
| | Q223K_ | KFPAFAQLKADGARCFAKVRGDELDDVRLL |
| | L231A_ | SRAGNEYLGLDLLKEELIKMTAEARQIHPE |
| | L293E_ | GVLIDGELVYHEKVKKEPEGADFLFDAYPE |
| | E440K | NSKAKEFAEVAESRTASNGIANKSLKGTIS |
| | | EKEAQCMKFQVWDYVPLVEIYSEPAFRLKY |
| | | DVRFSKLEQMTSGYDKVILIENQVVNNLDE |
| | | AKVIYKKYIDQGLEGIILKNIDGLWENARS |
| | | KNLYKFKEVIDVDLKIVGIYPHRKDPTKAG |
| | | GFILESECGKIKVNAGSGLKDKAGVKSHEL |
| | | DRTRIMENQNYYIGKILECKCNGWLKSDGR |
| | | TDYVKLFLPIAIRLREDKTKANTFEDVFGD |
| | | FHEVTGL |
| 3 | wt_ | MILKILNEIASIGSTKQKQAILEKNKDNEL |
| | E88K_ | LKRVYRLTYSRGLQYYIKKWPKPGIATQSF |
| | V119R_ | GMLTLTDMLDFIEFTLATRKLTGNAAIKEL |
| | Q223K_ | TGYITDGKKDDVEVLRRVMMRDLECGASRS |
| | L293E_ | IANKVWPGLIPEQPQMLASSYDEKGINKNI |
| | V372I_ | KFPAFAQLKADGARCFAEVRGDELDDVRLL |
| | E483K | SRAGNEYLGLDLLKEELIKMTAEARQIHPE |
| | | GVLIDGELVYHEKVKKEPEGLDFLFDAYPE |
| | | NSKAKEFAEVAESRTASNGIANKSLKGTIS |
| | | EKEAQCMKFQVWDYVPLVEIYSEPAFRLKY |
| | | DVRFSKLEQMTSGYDKVILIENQVVNNLDE |
| | | AKVIYKKYIDQGLEGIILKNIDGLWENARS |
| | | KNLYKFKEVIDIDLKIVGIYPHRKDPTKAG |
| | | GFILESECGKIKVNAGSGLKDKAGVKSHEL |
| | | DRTRIMENQNYYIGKILECECNGWLKSDGR |
| | | TDYVKLFLPIAIRLREDKTKANTFEDVFGD |
| | | FHKVTGL |

All sequences were in some instances expressed with a His6 tag (LEHHHHHH) for purification purposes at the C-terminus.

TABLE 2
Enzyme DNA Sequences

| SEQ | | |
|---|---|---|
| ID | | |
| NO | Name | DNA Sequence |
| 4 | WT_ | ATGATTCTTAAGATTCTGAATGAAATCGCG |
| | T4_ | AGCATCGGTAGCACGAAGCAGAAACAGGCC |
| | Ligase | ATCCTTGAAAAAAATAAAGACAACGAACTC |
| | | TTGAAGCGCGTCTACCGTCTTACCTACAGC |
| | | CGCGGTCTGCAGTACTATATTAAAAAATGG |
| | | CCAAAACCGGGAATCGCAACTCAATCGTTC |
| | | GGCATGCTTACCCTCACCGATATGCTGGAC |
| | | TTTATCGAGTTTACCTTGGCTACCCGCAAA |
| | | CTGACTGGCAATGCCGCTATCGAAGAACTG |
| | | ACTGGATACATTACGGATGGTAAAAAAGAT |
| | | GATGTCGAAGTGTTGCGGCGTGTCATGATG |
| | | CGCGATCTGGAGTGTGGCGCATCGGTGAGC |
| | | ATCGCAAACAAGGTTTGGCCTGGATTAATT |
| | | CCGGAACAGCCACAGATGCTTGCTTCATCT |
| | | TACGATGAGAAAGGCATTAACAAAAATATC |
| | | AAATTTCCAGCCTTTGCGCAACTGAAAGCA |
| | | GACGGCGCTCGTTGTTTCGCGGAAGTCCGC |
| | | GGTGATGAATTGGATGATGTCCGTCTGCTC |
| | | AGCCGCGCAGGGAACGAATATCTGGGCTTG |
| | | GACTTGCTGAAAGAGGAACTGATTAAGATG |
| | | ACCGCCGAAGCACGCCAGATCCACCCAGAA |
| | | GGCGTCCTGATTGATGGTGAATTGGTCTAC |
| | | CATGAACAGGTGAAAAAAGAACCGGAAGGC |
| | | TTAGATTTTCTCTTTGACGCATATCCGGAA |
| | | AACTCGAAAGCTAAAGAATTCGCTGAAGTT |
| | | GCCGAAAGCCGGACGGCGAGCAACGGTATC |
| | | GCGAACAAAAGCTTGAAAGGCACGATCAGC |
| | | GAGAAGGAGGCACAGTGTATGAAGTTTCAG |
| | | GTGTGGGATTATGTTCCGCTTGTAGAGATT |
| | | TATTCGTTACCCGCCTTCCGTTTAAAGTAC |
| | | GATGTTCGCTTTAGCAAACTTGAACAGATG |
| | | ACGAGCGGATATGACAAAGTGATTCTCATC |
| | | GAAAATCAGGTGGTCAACAATCTGGACGAG |
| | | GCCAAAGTGATCTATAAAAAATATATCGAC |
| | | CAGGGGCTTGAAGGTATTATTTTGAAAAAC |
| | | ATCGACGGCTTGTGGGAAAACGCGCGGAGC |
| | | AAAAATCTGTATAAATTTAAAGAGGTTATC |
| | | GACGTGGACTTAAAGATCGTTGGAATTTAC |
| | | CCGCATCGGAAAGACCCGACGAAAGCGGGT |
| | | GGCTTTATCCTGGAGTCTGAATGCGGCAAA |
| | | ATTAAAGTGAATGCTGGCTCTGGACTGAAA |
| | | GACAAAGCCGGTGTAAAAAGTCATGAGCTG |
| | | GATCGTACTCGCATTATGGAAAATCAGAAT |
| | | TATTATATTGGTAAAATCTTGGAATGTGAG |
| | | TGCAACGGCTGGCTGAAATCGGACGGGCGT |
| | | ACCGATTACGTGAAACTGTTCTTGCCGATT |
| | | GCTATTCGGTTACGCGAAGACAAAACTAAA |
| | | GCTAATACCTTCGAAGATGTGTTCGGCGAT |
| | | TTCCACGAGGTGACCGGCCTT |
| 5 | wt_ | ATGATTCTTAAGATTCTGAATGAAATCGCG |
| | E88K_ | AGCATCGGTAGCACGAAGCAGAAACAGGCC |
| | V119R | ATCCTTGAAAAAAATAAAGACAACGAACTC |
| | G128K_ | TTGAAGCGCGTCTACCGTCTTACCTACAGC |
| | E168K_ | CGCGGTCTGCAGTACTATATTAAAAAATGG |
| | Q223K_ | CCAAAACCGGGAATCGCAACTCAATCGTTC |
| | L231A_ | GGCATGCTTACCCTCACCGATATGCTGGAC |
| | L293E | TTTATCGAGTTTACCTTGGCTACCCGCAAA |
| | E440K | CTGACTGGCAATGCCGCTATCAAAGAACTG |
| | | ACTGGATACATTACGGATGGTAAAAAAGAT |
| | | GATGTCGAAGTGTTGCGGCGTGTCATGATG |
| | | CGCGATCTGGAGTGTGGCGCATCGCGTAGC |
| | | ATCGCAAACAAGGTTTGGCCTAAATTAATT |
| | | CCGGAACAGCCACAGATGCTTGCTTCATCT |
| | | TACGATGAGAAAGGCATTAACAAAAATATC |
| | | AAATTTCCAGCCTTTGCGCAACTGAAAGCA |
| | | GACGGCGCTCGTTGTTTCGCGAAAGTCCGC |
| | | GGTGATGAATTGGATGATGTCCGTCTGCTC |
| | | AGCCGCGCAGGGAACGAATATCTGGGCTTG |
| | | GACTTGCTGAAAGAGGAACTGATTAAGATG |
| | | ACCGCCGAAGCACGCCAGATCCACCCAGAA |
| | | GGCGTCCTGATTGATGGTGAATTGGTCTAC |
| | | CATGAAAAAGTGAAAAAAGAACCGGAAGGC |
| | | GCAGATTTTCTCTTTGACGCATATCCGGAA |
| | | AACTCGAAAGCTAAAGAATTCGCTGAAGTT |
| | | GCCGAAAGCCGGACGGCGAGCAACGGTATC |
| | | GCGAACAAAAGCTTGAAAGGCACGATCAGC |
| | | GAGAAGGAGGCACAGTGTATGAAGTTTCAG |
| | | GTGTGGGATTATGTTCCGCTTGTAGAGATT |
| | | TATTCGGAACCCGCCTTCCGTTTAAAGTAC |
| | | GATGTTCGCTTTAGCAAACTTGAACAGATG |
| | | ACGAGCGGATATGACAAAGTGATTCTCATC |
| | | GAAAATCAGGTGGTCAACAATCTGGACGAG |
| | | GCCAAAGTGATCTATAAAAAATATATCGAC |
| | | CAGGGGCTTGAAGGTATTATTTTGAAAAAC |
| | | ATCGACGGCTTGTGGGAAAACGCGCGGAGC |
| | | AAAAATCTGTATAAATTTAAAGAGGTTATC |
| | | GACGTGGACTTAAAGATCGTTGGAATTTAC |
| | | CCGCATCGGAAAGACCCGACGAAAGCGGGT |
| | | GGCTTTATCCTGGAGTCTGAATGCGGCAAA |
| | | ATTAAAGTGAATGCTGGCTCTGGACTGAAA |
| | | GACAAAGCCGGTGTAAAAAGTCATGAGCTG |
| | | GATCGTACTCGCATTATGGAAAATCAGAAT |
| | | TATTATATTGGTAAAATCTTGGAATGTAAA |
| | | TGCAACGGCTGGCTGAAATCGGACGGGCGT |
| | | ACCGATTACGTGAAACTGTTCTTGCCGATT |
| | | GCTATTCGGTTACGCGAAGACAAAACTAAA |
| | | GCTAATACCTTCGAAGATGTGTTCGGCGAT |
| | | TTCCACGAGGTGACCGGCCTT |
| 6 | wt_ | ATGATTCTTAAGATTCTGAATGAAATCGCG |
| | E88K_ | AGCATCGGTAGCACGAAGCAGAAACAGGCC |
| | V119R_ | ATCCTTGAAAAAAATAAAGACAACGAACTC |
| | Q223K_ | TTGAAGCGCGTCTACCGTCTTACCTACAGC |
| | L293E_ | CGCGGTCTGCAGTACTATATTAAAAAATGG |
| | V372I_ | CCAAAACCGGGAATCGCAACTCAATCGTTC |
| | E483K | GGCATGCTTACCCTCACCGATATGCTGGAC |
| | | TTTATCGAGTTTACCTTGGCTACCCGCAAA |
| | | CTGACTGGCAATGCCGCTATCAAAGAACTG |
| | | ACTGGATACATTACGGATGGTAAAAAAGAT |
| | | GATGTCGAAGTGTTGCGGCGTGTCATGATG |
| | | CGCGATCTGGAGTGTGGCGCATCGCGTAGC |
| | | ATCGCAAACAAGGTTTGGCCTGGATTAATT |
| | | CCGGAACAGCCACAGATGCTTGCTTCATCT |
| | | TACGATGAGAAAGGCATTAACAAAAATATC |
| | | AAATTTCCAGCCTTTGCGCAACTGAAAGCA |
| | | GACGGCGCTCGTTGTTTCGCGGAAGTCCGC |
| | | GGTGATGAATTGGATGATGTCCGTCTGCTC |
| | | AGCCGCGCAGGGAACGAATATCTGGGCTTG |
| | | GACTTGCTGAAAGAGGAACTGATTAAGATG |
| | | ACCGCCGAAGCACGCCAGATCCACCCAGAA |
| | | GGCGTCCTGATTGATGGTGAATTGGTCTAC |
| | | CATGAAAAAGTGAAAAAAGAACCGGAAGGC |
| | | TTAGATTTTCTCTTTGACGCATATCCGGAA |
| | | AACTCGAAAGCTAAAGAATTCGCTGAAGTT |
| | | GCCGAAAGCCGGACGGCGAGCAACGGTATC |
| | | GCGAACAAAAGCTTGAAAGGCACGATCAGC |
| | | GAGAAGGAGGCACAGTGTATGAAGTTTCAG |
| | | GTGTGGGATTATGTTCCGCTTGTAGAGATT |
| | | TATTCGGAACCCGCCTTCCGTTTAAAGTAC |
| | | GATGTTCGCTTTAGCAAACTTGAACAGATG |
| | | ACGAGCGGATATGACAAAGTGATTCTCATC |
| | | GAAAATCAGGTGGTCAACAATCTGGACGAG |
| | | GCCAAAGTGATCTATAAAAAATATATCGAC |
| | | CAGGGGCTTGAAGGTATTATTTTGAAAAAC |
| | | ATCGACGGCTTGTGGGAAAACGCGCGGAGC |
| | | AAAAATCTGTATAAATTTAAAGAGGTTATC |
| | | GACATTGACTTAAAGATCGTTGGAATTTAC |
| | | CCGCATCGGAAAGACCCGACGAAAGCGGGT |
| | | GGCTTTATCCTGGAGTCTGAATGCGGCAAA |
| | | ATTAAAGTGAATGCTGGCTCTGGACTGAAA |
| | | GACAAAGCCGGTGTAAAAAGTCATGAGCTG |
| | | GATCGTACTCGCATTATGGAAAATCAGAAT |
| | | TATTATATTGGTAAAATCTTGGAATGTGAG |
| | | TGCAACGGCTGGCTGAAATCGGACGGGCGT |
| | | ACCGATTACGTGAAACTGTTCTTGCCGATT |
| | | GCTATTCGGTTACGCGAAGACAAAACTAAA |
| | | GCTAATACCTTCGAAGATGTGTTCGGCGAT |
| | | TTCCACAAAGTGACCGGCCTT |

All sequences were in some instances expressed with a His6 tag (CTCGAGCACCACCACCACCACCAC) for purification purposes at the C-terminus.

An enzyme provided herein may comprise a sequence having homology or similarity with SEQ ID NO.: 1. In some instances, an enzyme does not comprise SEQ ID NO.: 1. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 1. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 1. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 1. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 1. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 1.

An enzyme provided herein may comprise a sequence having homology or similarity with SEQ ID NO.: 2. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 2. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 2. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 2. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 2. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 2.

An enzyme provided herein may comprise a sequence having homology or similarity with SEQ ID NO.: 3. In some instances, an enzyme provided herein comprises at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 3. In some instances, at least 10 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 3. In some instances, at least 50 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 3. In some instances, at least 100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 3. In some instances, 20-100 contiguous amino acids of an enzyme provided herein comprise at least about 50%, at least about 60%, at least about 70%, at least about 80%, at least about 85%, at least about 90%, at least about 95%, at least about 97%, at least about 98%, at least about 99%, at least about 99.5%, or more similarity with SEQ ID NO.: 3.

An enzyme provided herein may comprise a sequence having homology or similarity and mutations at one or more amino acid positions. In some instances, an enzyme comprises a mutation at one or more of positions selected from 88, 91, 119, 128, 168, 223, 231, 293, 372, 440, 448, or 483 and at least 95% similarity to SEQ ID NO.: 1. In some instances, an enzyme comprises a mutation at two or more of positions selected from 88, 91, 119, 128, 168, 223, 231, 293, 372, 440, 448, or 483 and at least 90% similarity to SEQ ID NO.: 1. In some instances, an enzyme comprises a mutation at three or more of positions selected from 88, 91, 119, 128, 168, 223, 231, 293, 372, 440, 448, or 483 and at least 90% similarity to SEQ ID NO.: 1. In some instances, an enzyme comprises a mutation at four or more of positions selected from 88, 91, 119, 128, 168, 223, 231, 293, 372, 440, 448, or 483 and at least 90% similarity to SEQ ID NO.: 1. In some instances, an enzyme comprises a mutation at five or more of positions selected from 88, 91, 119, 128, 168, 223, 231, 293, 372, 440, 448, or 483 and at least 90% similarity to SEQ ID NO.: 1. In some instances, an enzyme comprises a mutation at six or more of positions selected from 88, 91, 119, 128, 168, 223, 231, 293, 372, 440, 448, or 483 and at least 90% similarity to SEQ ID NO.: 1. In some instances, an enzyme comprises a mutation at seven or more of positions selected from 88, 91, 119, 128, 168, 223, 231, 293, 372, 440, 448, or 483 and at least 90% similarity to SEQ ID NO.: 1. In some instances, an enzyme comprises a mutation at eight or more of positions selected from 88, 91, 119, 128, 168, 223, 231, 293, 372, 440, 448, or 483 and at least 90% similarity to SEQ ID NO.: 1. In some instances, an enzyme comprises a mutation at nine or more of positions selected from 88, 91, 119, 128, 168, 223, 231, 293, 372, 440, 448, or 483 and at least 90% similarity to SEQ ID NO.: 1. In some instances, an enzyme comprises a mutation at ten or more of positions selected from 88, 91, 119, 128, 168, 223, 231, 293, 372, 440, 448, or 483 and at least 90% similarity to SEQ ID NO.: 1.

In some instances, an enzyme comprises a mutation at one or more of positions selected from 88, 91, 119, 128, 168, 223, 231, 293, 372, 440, 448, or 483 and at least 80% similarity to SEQ ID NO.: 1. In some instances, an enzyme comprises a mutation at two or more of positions selected from 88, 91, 119, 128, 168, 223, 231, 293, 372, 440, 448, or 483 and at least 80% similarity to SEQ ID NO.: 1. In some instances, an enzyme comprises a mutation at three or more of positions selected from 88, 91, 119, 128, 168, 223, 231, 293, 372, 440, 448, or 483 and at least 80% similarity to SEQ ID NO.: 1. In some instances, an enzyme comprises a mutation at four or more of positions selected from 88, 91, 119, 128, 168, 223, 231, 293, 372, 440, 448, or 483 and at least 80% similarity to SEQ ID NO.: 1. In some instances, an enzyme comprises a mutation at five or more of positions selected from 88, 91, 119, 128, 168, 223, 231, 293, 372, 440, 448, or 483 and at least 80% similarity to SEQ ID NO.: 1. In some instances, an enzyme comprises a mutation at six or more of positions selected from 88, 91, 119, 128, 168, 223, 231, 293, 372, 440, 448, or 483 and at least 80% similarity to SEQ ID NO.: 1. In some instances, an enzyme comprises a mutation at seven or more of positions selected from 88, 91, 119, 128, 168, 223, 231, 293, 372, 440, 448, or 483 and at least 80% similarity to SEQ ID NO.: 1. In some instances, an enzyme comprises a mutation at eight or more of positions selected from 88, 91, 119, 128, 168, 223, 231, 293, 372, 440, 448, or 483 and at least 80% similarity to SEQ ID NO.: 1. In some instances, an enzyme comprises a mutation at nine or more of positions selected from 88, 91, 119, 128, 168, 223, 231, 293, 372, 440, 448, or 483 and at least 80% similarity to SEQ ID NO.: 1. In some instances, an enzyme comprises a mutation at ten or more of positions selected from 88, 91, 119, 128, 168, 223, 231, 293, 372, 440, 448, or 483 and at least 80% similarity to SEQ ID NO.: 1.

An enzyme provided herein may comprise specific amino acid mutations. In some instances, an enzyme comprises one or more mutations selected from E88K, T91M, V119R, G128K, E168K, Q223K, L231A, L293E, V372I, E440K, D448W, D448P, and E483K relative to SEQ ID NO.: 1. In some instances, an enzyme comprises two or more mutations selected from E88K, T91M, V119R, G128K, E168K, Q223K, L231A, L293E, V372I, E440K, D448W, D448P, and E483K relative to SEQ ID NO.: 1. In some instances, an enzyme comprises three or more mutations selected from E88K, T91M, V119R, G128K, E168K, Q223K, L231A, L293E, V372I, E440K, D448W, D448P, and E483K relative to SEQ ID NO.: 1. In some instances, an enzyme comprises five or more mutations selected from E88K, T91M, V119R, G128K, E168K, Q223K, L231A, L293E, V372I, E440K, D448W, D448P, and E483K relative to SEQ ID NO.: 1. In some instances, an enzyme comprises one or more mutations selected from E88K, V119R, G128K, E168K, Q223K, L231A, L293E, and E440K relative to SEQ ID NO.: 1. In some instances, an enzyme comprises one or more mutations selected from E88K, V119R, Q223K, L293E, V372I, and E483K relative to SEQ ID NO.: 1. In some instances, an enzyme comprises two or more mutations selected from E88K, V119R, G128K, E168K, Q223K, L231A, L293E, and E440K relative to SEQ ID NO.: 1. In some instances, an enzyme comprises two or more mutations selected from E88K, V119R, Q223K, L293E, V372I, and E483K relative to SEQ ID NO.: 1. In some instances, an enzyme comprises four or more mutations selected from E88K, V119R, Q223K, L293E, V372I, and E483K relative to SEQ ID NO.: 1. In some instances, an enzyme comprises two or more mutations selected from E88K, V119R, Q223K, L293E, E440K, and D448W relative to SEQ ID NO.: 1. In some instances, an enzyme comprises three or more mutations selected from E88K, V119R, Q223K, L293E, E440K, and D448W relative to SEQ ID NO.: 1. In some instances, an enzyme comprises four or more mutations selected from E88K, V119R, Q223K, L293E, E440K, and D448W relative to SEQ ID NO.: 1. In some instances, an enzyme comprises two or more mutations selected from E88K, T91M, V119R, G128K, Q223K, L293E, and E440K relative to SEQ ID NO.: 1. In some instances, an enzyme comprises three or more mutations selected from 88K, T91M, V119R, G128K, Q223K, L293E, and E440K relative to SEQ ID NO.: 1. In some instances, an enzyme comprises four or more mutations selected from 88K, T91M, V119R, G128K, Q223K, L293E, and E440K relative to SEQ ID NO.: 1.

### Enzyme Optimization

Described herein are methods and systems of in-silico library design. For example, an enzyme or enzyme fragment sequence is used as input. Any enzyme sequence is in some instances used for input in to the methods and systems described herein. A database comprising known mutations from an organism is queried, and a library of sequences comprising combinations of these mutations are generated. In some instances, specific mutations or combinations of mutations are excluded from the library (e.g., known immunogenic sites, structure sites, etc.). In some instances, specific sites in the input sequence are systematically replaced with histidine, aspartic acid, glutamic acid, or combinations thereof. In some instances, the maximum or minimum number of mutations allowed for each region of an enzyme are specified. Mutations in some instances are described relative to the input sequence or the input sequence's corresponding germline sequence. For example, sequences generated by the optimization comprise at least 1, at least 2, at least 3, at least 4, at least 5, at least 6, at least 7, at least 8, at least 9, at least 10, at least 11, at least 12, at least 13, at least 14, at least 15, at least 16, or more than 16 mutations from the input sequence. In some instances, sequences generated by the optimization comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, or no more than 18 mutations from the input sequence. In some instances, sequences generated by the optimization comprise about 1, about 2, about 3, about 4, about 5, about 6, about 7, about 8, about 9, about 10, about 11, about 12, about 13, about 14, about 15, about 16, or about 18 mutations relative to the input sequence. In-silico enzyme libraries are in some instances synthesized, assembled, and/or enriched for desired sequences.

The germline sequences corresponding to an input sequence may also be modified to generate sequences in a library. For example, sequences generated by the optimization methods described herein comprise at least 1, at least 2, at least 3, at least 4, at least 5, at least 6, at least 7, at least 8, at least 9, at least 10, at least 11, at least 12, at least 13, at least 14, at least 15, at least 16, or more than 16 mutations from the germline sequence. In some instances, sequences generated by the optimization comprise no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16 or no more than 18 mutations from the germline sequence. In some instances, sequences generated by the optimization comprise about 1, about 2, about 3, about 4, about 5, about 6, about 7, about 8, about 9, about 10, about 11, about 12, about 13, about 14, about 15, about 16, or about 18 mutations relative to the germline sequence.

### Machine Learning

The data from preprocessing operations, as described herein, may be fed into one or more machine learning (ML) algorithms for identifying a library comprising one or more candidates with high affinity to a target and/or functional activity. In some embodiments, the one or more candidates comprise one or more sequences encoding for an enzyme. In some examples, the library may be a synthetic library. In some embodiments, the ML algorithms may be integrated into a computational pipeline for intelligent decision making and/or experimental validation. In some embodiments, the one or more ML algorithms may be supervised, semi-supervised, or unsupervised for training to identify anomalies. In some embodiments, the one or more ML algorithms may perform classification or clustering to identify anomalies or attacks. In some embodiments, the one or more ML algorithms may comprise classical ML algorithms for performing clustering to identify outliers. Classical ML algorithms may comprise of algorithms that learn from existing observations (i.e., known features) to predict outputs. In some cases, the classical ML algorithms for performing clustering may be K-means clustering, mean-shift clustering, density-based spatial clustering of applications with noise (DBSCAN), expectation-maximization (EM) clustering (e.g., using Gaussian mixture models (GMM)), agglomerative hierarchical clustering, or a combination thereof. In some embodiments, the one or more ML algorithms may comprise classical ML algorithms for classification. In some cases, the classical ML algorithms may comprise logistic regression, naïve Bayes, K-nearest neighbors, random forests or decision trees, gradient boosting, support vector machines (SVMs), or a combination thereof. In some embodiments, the one or more ML algorithm may employ deep learning. A deep learning algorithm may comprise of an algorithm that learns by extracting new features to predict outputs. The deep learning algorithm may comprise of layers, which may comprise a neural network.

### Expression Systems

Provided herein are libraries comprising nucleic acids encoding for enzymes, wherein the libraries have improved specificity, stability, expression, folding, or downstream activity. In some instances, libraries described herein are used for screening and analysis.

Provided herein are libraries comprising nucleic acids encoding for enzymes, wherein the nucleic acid libraries are used for screening and analysis. In some instances, screening and analysis comprises in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, a rat, a rabbit, a primate (e.g., a monkey or an ape), a fish, a worm, a bird, a chicken, a camelid, a cat, a dog, a horse, a cow, a sheep, a goat, a frog, or an insect.

Nucleic acid libraries described herein may be screened for various pharmacological or pharmacokinetic properties. In some instances, the libraries are screened using in vitro assays, in vivo assays, or ex vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Provided herein are nucleic acid libraries, wherein the nucleic acid libraries may be expressed in a vector. Expression vectors for inserting nucleic acid libraries disclosed herein may comprise eukaryotic or prokaryotic expression vectors. Exemplary expression vectors include, without limitation, mammalian expression vectors: pSF-CMV-NEO-NH2-PPT-3XFLAG, pSF-CMV-NEO—COOH-3XFLAG, pSF-CMV—PURO-NH2-GST-TEV, pSF-OXB20—COOH)-TEV-FLAG (R)-6His, pCEP4 pDEST27, pSF-CMV-Ub-KrYFP, pSF-CMV-FMDV-daGFP, pEFla-mCherry-N1 Vector, pEF1a-tdTomato Vector, pSF-CMV-FMDV-Hygro, pSF-CMV-PGK-Puro, pMCP-tag (m), and pSF-CMV—PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. In some instances, the vector is pcDNA3 or pcDNA3.1.

Described herein are nucleic acid libraries that are expressed in a vector to generate a construct comprising an enzyme. In some instances, a size of the construct varies. In some instances, the construct comprises at least or about 500, at least or about 600, at least or about 700, at least or about 800, at least or about 900, at least or about 1000, at least or about 1100, at least or about 1300, at least or about 1400, at least or about 1500, at least or about 1600, at least or about 1700, at least or about 1800, at least or about 2000, at least or about 2400, at least or about 2600, at least or about 2800, at least or about 3000, at least or about 3200, at least or about 3400, at least or about 3600, at least or about 3800, at least or about 4000, at least or about 4200, at least or about 4400, at least or about 4600, at least or about 4800, at least or about 5000, at least or about 6000, at least or about 7000, at least or about 8000, at least or about 9000, at least or about 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for enzymes, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

Provided herein are libraries comprising nucleic acids encoding for enzymes (e.g., ligases). Enzymes described herein allow for improved stability for a range of active site encoding sequences. In some instances, the active site encoding sequences are determined by interactions between the substrate and the catalytically active site of an enzyme.

Sequences of active sites based on surface interactions between a ligand/substrate and an enzyme described herein are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (www.uniprot.org/), and IUPHAR/BPS Guide to PHARMACOLOGY (guidetopharmacology.org/).

Described herein are active sites designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, camelid, fish, fly, or human. In some instances, homologous sequences are identified in the same organism, across individuals.

Following identification of active sites, libraries comprising nucleic acids encoding for the active sites may be generated. In some instances, libraries of active sites comprise sequences of active sites designed based on conformational ligand/substrate interactions. Libraries of active sites may be translated to generate protein libraries. In some instances, libraries of active sites are translated to generate peptide libraries, immunoglobulin libraries, derivatives thereof, or combinations thereof. In some instances, libraries of active sites are translated to generate protein libraries that are further modified to generate peptidomimetic libraries. In some instances, libraries of active sites are translated to generate protein libraries that are used to generate small molecules.

Methods described herein provide for synthesis of libraries of active sites comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of active sites comprise varied nucleic acids collectively encoding variations at multiple positions. In some instances, the variant library comprises sequences encoding for variation of at least a single codon in an active site. In some instances, the variant library comprises sequences encoding for variation of multiple codons in an active site. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the active sites, wherein the libraries comprise sequences encoding for variation of length of the active sites. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of active sites, enzymes may be designed and synthesized to comprise the active sites. Enzymes comprising active sites may be designed based on binding, specificity, stability, expression, folding, or downstream activity.

Methods described herein provide for synthesis of a library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the library comprises varied nucleic acids collectively encoding variations at multiple positions. In some instances, the variant library comprises sequences encoding for variation of at least a single codon in an active site. For example, at least one single codon of the enzyme is varied. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of a library of nucleic acids each encoding for a predetermined variant of at least one predetermined reference nucleic acid sequence, wherein the library comprises sequences encoding for variation of length of a domain in the enzyme. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following synthesis of enzyme libraries for screening and analysis. For example, libraries are assayed for library displayability, screening, and/or panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. In instances, libraries are assayed for ligase activity or stability.

### Variant Libraries

### Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 3 provides a listing of each codon possible (and the representative amino acid) for a variant site.

TABLE 3
List of codons and amino acids

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Iso | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |

| | | | | | | |
|---|---|---|---|---|---|---|
| Threonine | T | Thr | ACA | ACC | ACG | ACT |
| Valine | V | Va | GTA | GTC | GTG | GTT |

| Tryptophan | W | Trp | TGG | |
|---|---|---|---|---|
| Tyrosine | Y | Tyr | TAC | TAT |

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

### Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, an enzyme itself can be optimized using methods described herein. For example, to improve a specified function of an enzyme, a variant polynucleotide library encoding for a portion of the enzyme is designed and synthesized. A variant nucleic acid library for the enzyme can then be generated by processes described herein (e.g., PCR mutagenesis followed by insertion into a vector). The enzyme is then expressed in a production cell line and screened for enhanced activity. Example screens include examining modulation in binding affinity to a substrate, stability (e.g., heat, salt), or function (e.g., substrate scope, speed).

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites. In some instances, the one or more sites of variant codon sites may be adjacent. In some instances, the one or more sites of variant codon sites may not be adjacent and separated by 1, 2, 3, 4, 5, 6, 7, 8, 9, 10, or more codons. In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

### Sequencing

Enzymes provided herein may be used for a variety of downstream applications. In some instances, enzymes comprise ligases. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as a T4 DNA polymerase or variant there, klenow enzyme, and T4 polynucleotide kinase in an appropriate buffer. A nucleotide overhang to facilitate ligation to adapters is added, in some instances with 3′ to 5′ exo minus klenow fragment and dATP.

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters.

A plurality of nucleic acids (i.e. genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification. SMSS is described in part in US Publication application Nos. 2006002471 I; 20060024678; 20060012793; 20060012784; and 20050100932.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors”, Nature, doi: 10.1038/nature03959; and well as in US Publication application Nos. 20020012930; 20030058629; 20030100102; 20030148344; 20040248161; 20050079510, 20050124022; and 20060078909.

In some instances, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication application Nos. 20040106130; 20030064398; 20030022207; and Constans, A., The Scientist 2003, 17 (13): 36. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication application No. 20030044781 and 2006/0078937. Overall such systems involve sequencing a target oligonucleotide molecule having a plurality of bases by the temporal addition of bases via a polymerization reaction that is measured on a molecule of oligonucleotide, i e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing {See e.g., Soni G V and Meller A. (2007) Clin Chem 53:1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327:78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

Provided herein are methods for preparing a nucleic acid library comprising one or more steps of providing one or more sample nucleic acids; contacting the one or more sample nucleic acids with a plurality of adapters and a T4 ligase variant described herein to form a nucleic acid sequencing library comprising adapter-ligated nucleic acids; and sequencing the nucleic acid library. In some instances, the sample nucleic acids comprise genomic fragments.

In some instances, the genomic fragments are obtained from cleavage of a genome. In some instances, the genomic fragments are obtained from amplification of a genome. In some instances the sample nucleic acids comprise cDNAs. In some instances the sample nucleic acids comprise cfDNAs. In some instances the method further comprises one or more steps to prepare nucleic acid library, such as end-repair, a-tailing, and amplification. In some instances the method further comprises enriching the nucleic acid library prior to sequencing.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: T4 Ligase High Throughput Assay

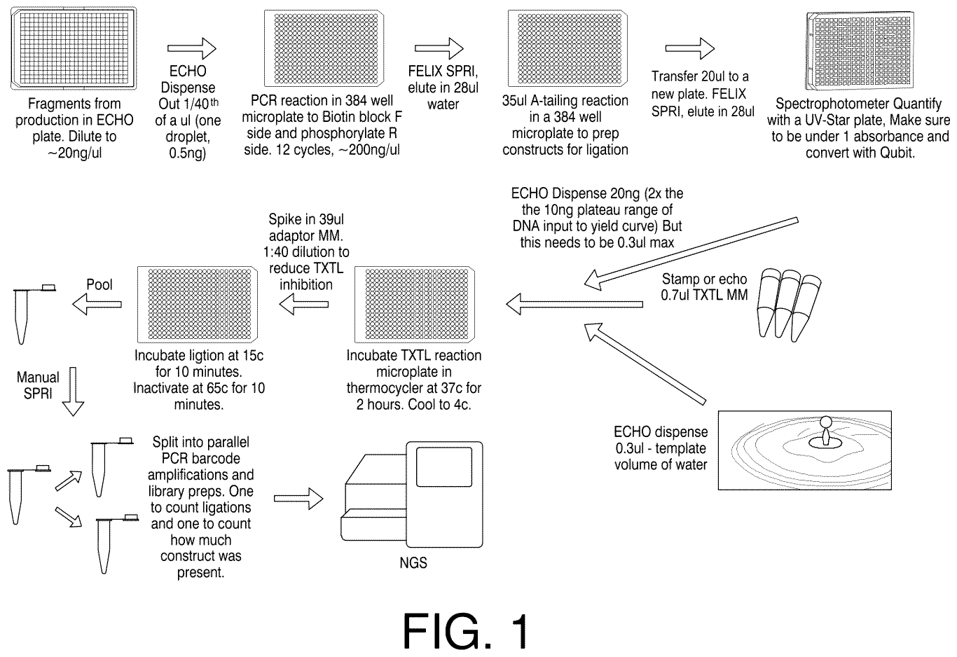

T4 variants were tested using the general protocol outlined in FIG. 1. An echo liquid handler system (Beckman) was used to dispense DNA encoding for T4 ligase variants into a 384 well plate. Each fragment was diluted to a concentration of 20 ng/microliter. 1/40^{th} of a microliter (one droplet, 0.5 ng) of each well was transferred to a new 384 well plate. PCR was then carried out in the 384 well microplate to biotin block the F side, and phosphorylate the R side. After amplification, 20 microliters was transferred to a new plate and Felix SPRI was used to isolate amplicons. Amplicons were then eluted in 28 microliters for spectrophotometer quantification. Concentrations were at least 67 ng/microliter. 20 ng (2X the 10 ng plateau range of DNA input to yield curve, 0.3 microliters) was then dispensed into a new plate, as well as a TXTL mastermix (NEB) for cell-free protein expression reaction. The plate containing the TXTL reactions was incubated in a thermocycler at 37 C for 2 hours, then cooled to 4C. Wells were then spiked with 39 microliters of adapter master mix at a 1:40 dilution to reduce TXTL inhibition. The ligation was incubated at 15C for 10 minutes, then inactivated at 65C for 10 minutes. Samples from each well were pooled, purified by manual SPRI beads, then split into parallel PCR barcode amplifications and library preparations (one to count ligations and one to count how much construct was present). Both pools were then sequenced.

### Example 2: T4 Ligase Optimization

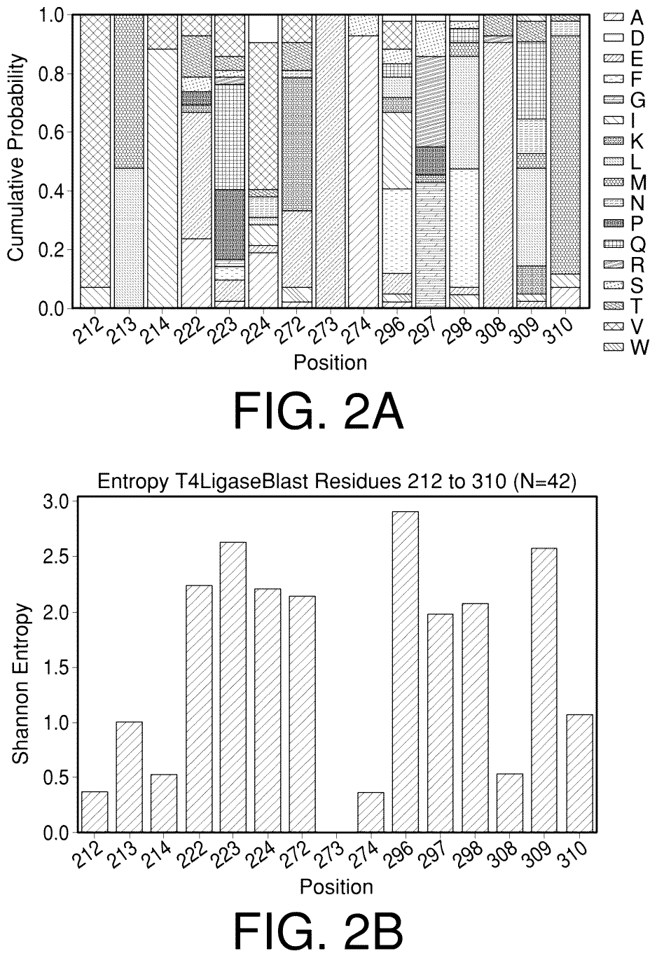

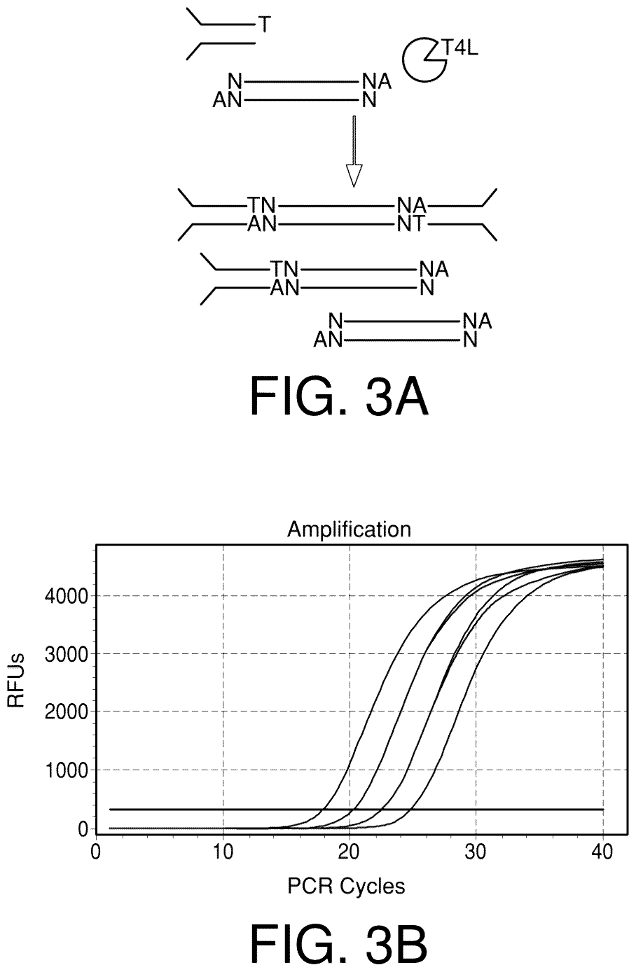

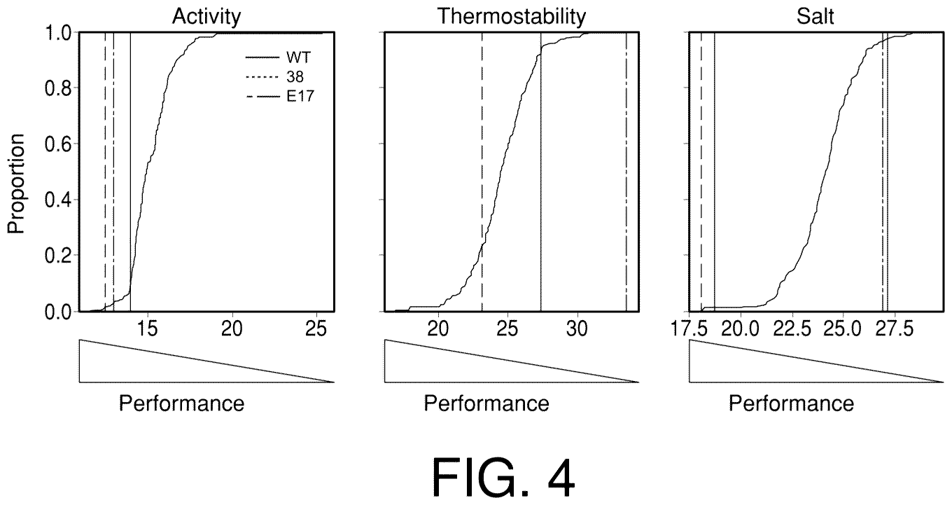

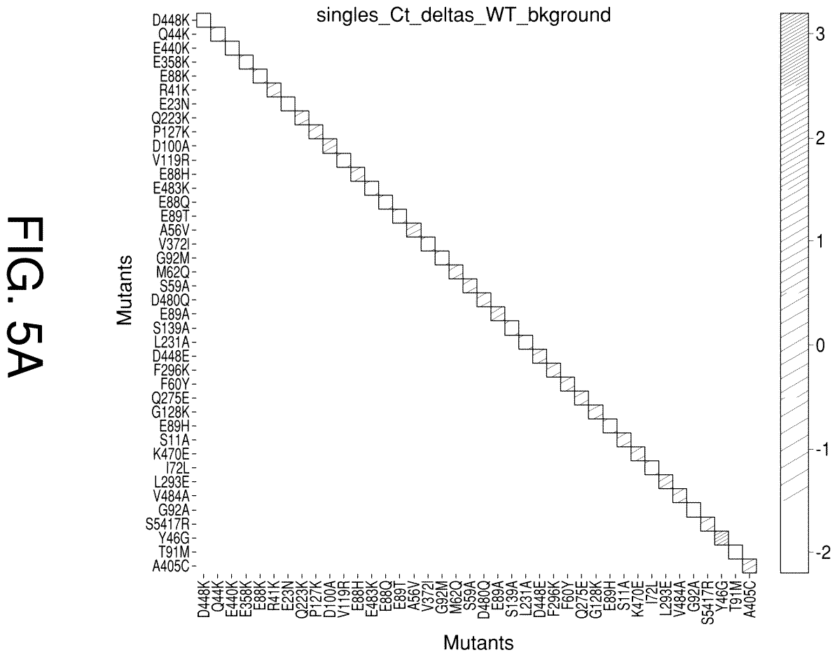

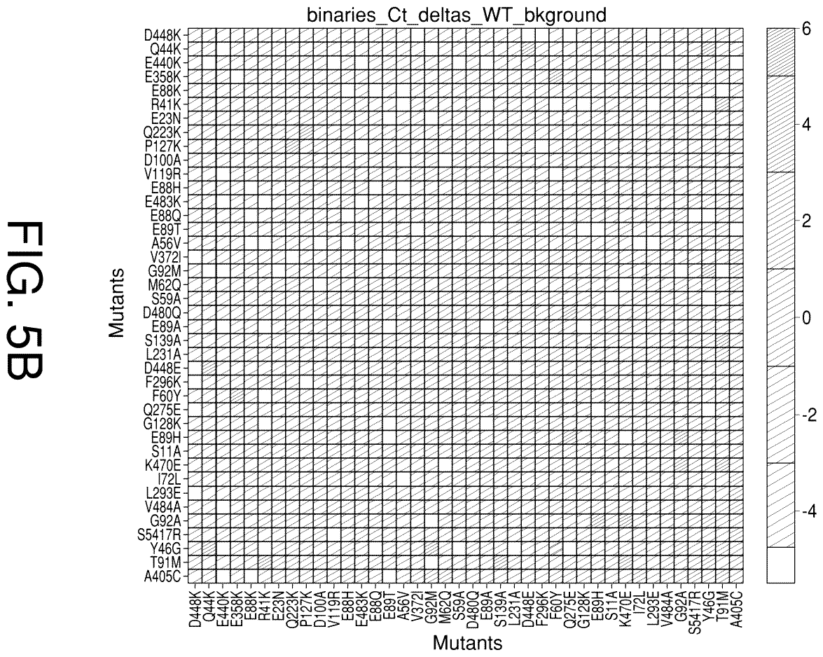

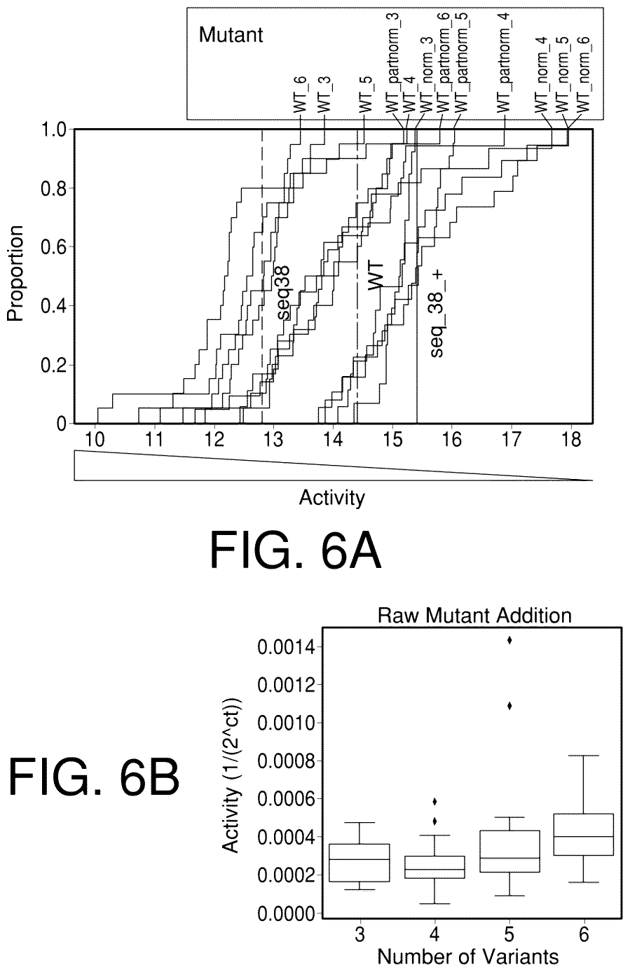

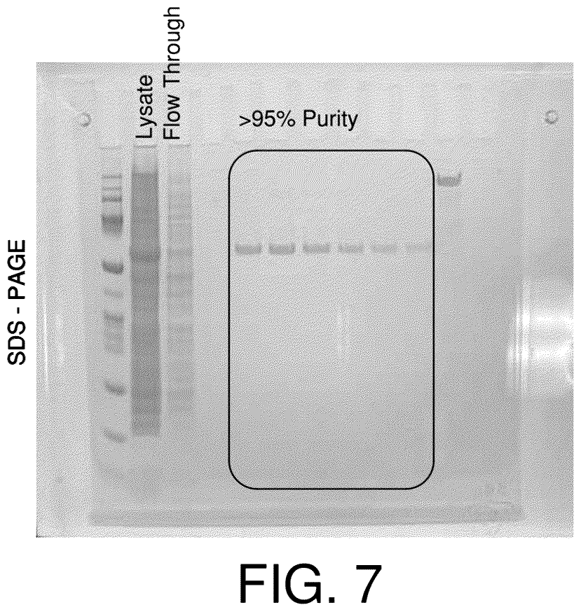

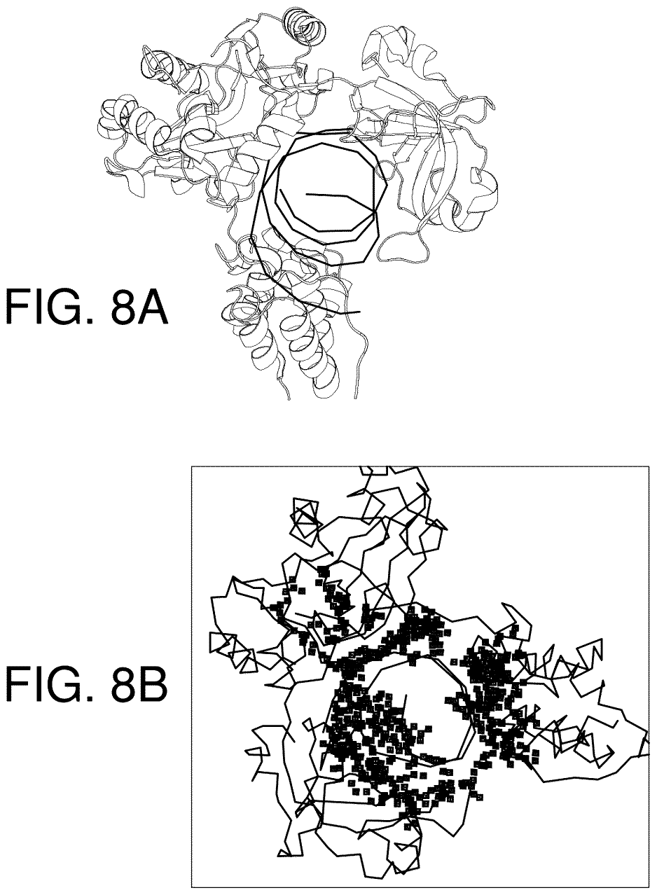

Following the general procedures of Example 1, multiple rounds of optimization/selection were used to generate T4 ligase variants. Variants from the wild type sequence (SEQ ID NO.: 1) were selected based in part on high entropy positions (FIGS. 2A-2B), and screened using a high output qPCR assay (FIGS. 3A-3B). In a first round, single variants were tested for ligation performance metrics including activity, thermostability, and salt tolerance (FIG. 4). In screening rounds 2/3, all binary combinations of mutants were evaluated for epistatic relationships (FIGS. 5A-5B). Raw additions were also used (FIGS. 6A-6B). Structural information was also fed into the design using an iterative process, including the location of lysine mutations near the DNA substrate (FIGS. 8A-8B). Beneficial lysine mutants were found clustered close to DNA contact regions. Locations near the DNA substrate were iteratively mutated to lysines to test for an activity improvement. Revised designs from this approach were expressed as His6-tagged constructs and subjected to molecular biology grade protein purification for evaluation in an NGS Assay with cfDNA performance comparison (FIG. 7) for rounds 6/7. Briefly, 10 ng input cfDNA (cfDNA Reference #104549, Twist Bioscience) was end-repaired/a-tailed (Mechanical Fragmentation #104177, Twist Bioscience), ligated with adapters (UDI Adapters #101308, Twist Bioscience) and variant ligase (250 ng), amplified (Mechanical Fragmentation #104177, Twist Bioscience), and sequenced on a NextSeq 500/550 instrument (Illumina). The standard ligation protocol used was 10×DNA ligase buffer (2 microliters), 40% PEG (2.5 microliters), adapters (1 microliter), ligase dilution (125 ng/microliter), ER/AT cfDNA sample (10 microliters), and water (2.5 microliters). The reaction was incubated using a Thermal Cycler (Heated Lid at 70° C.) with the program: 20C for 15 minutes, 65C for 10 minutes, and 4C hold. Experiments were each the result of two replicates, and included T4 wild-type as a control.

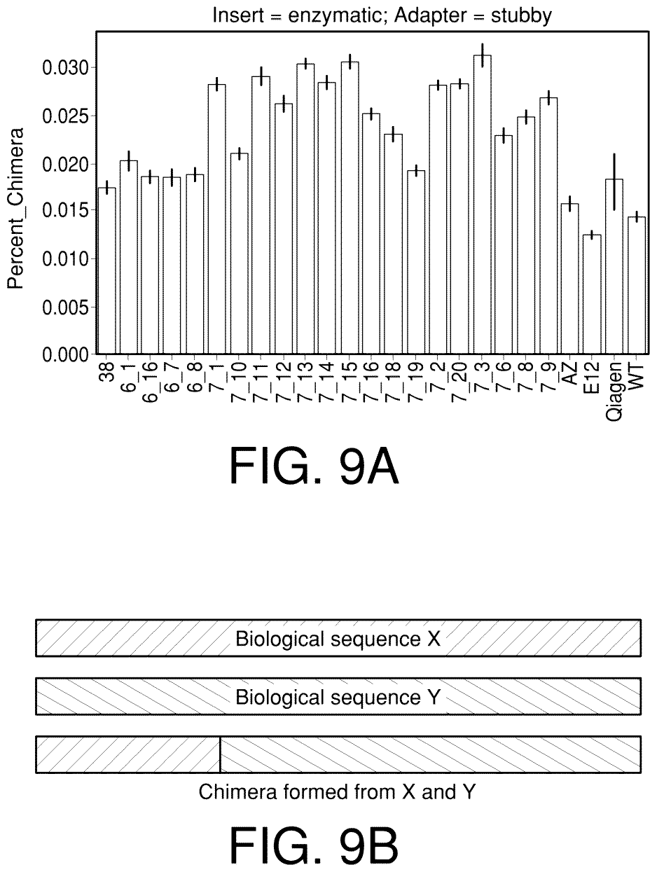

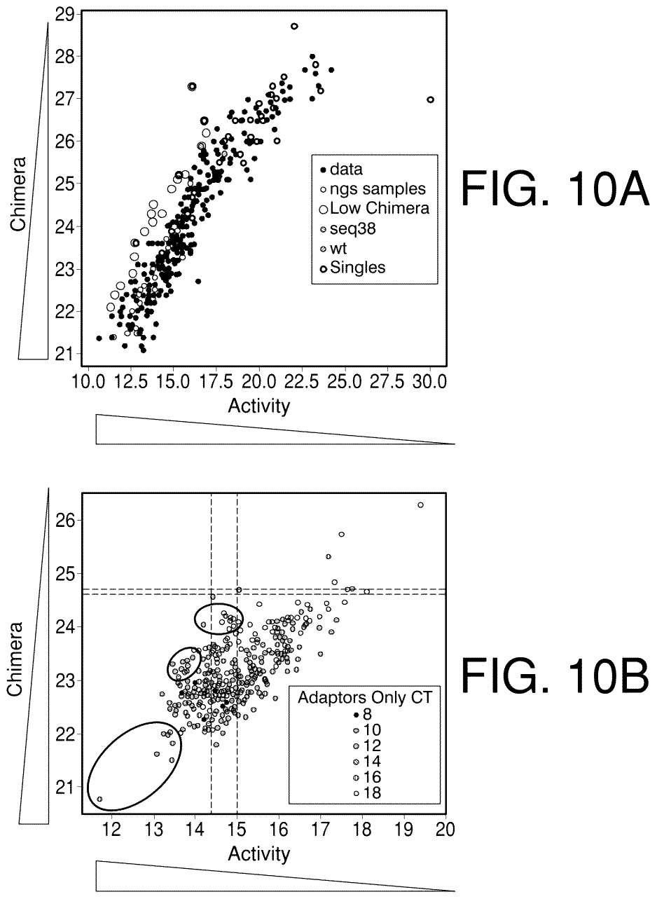

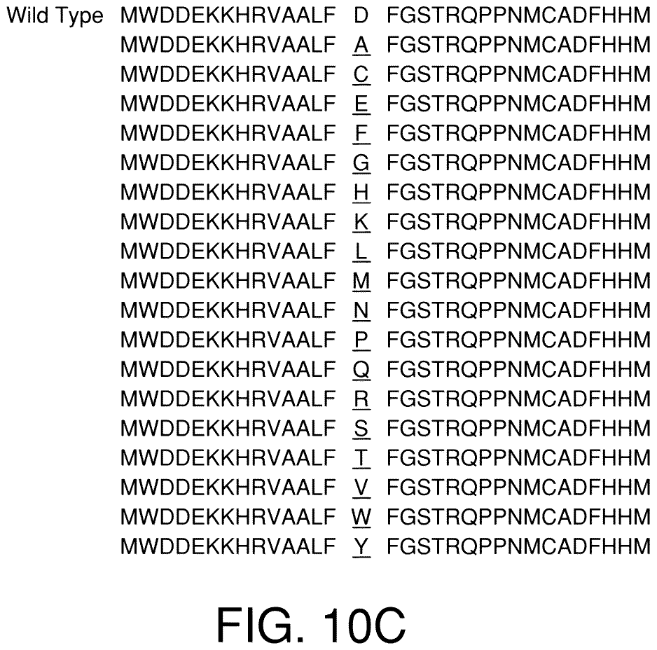

Pass filter reads were used to evaluate these constructs, as shown in FIG. 9A. Adding additional mutations introduced a negative phenotype into the protein (FIG. 9B). For round 8, an orthogonal assay was then conducted to determine chimeras by re-screening of Round 6 hits using site directed mutagenesis of chimera-prone sites (FIGS. 10A-10B).

Round 9 involved generation of single site mutations to select for variants that do not increase chimera (FIG. 10C), and hits were screened using the NGS assay described for rounds 6/7. Round nine involved 9× Design, 10× 384w plates, 24× assays using 47 purified ligases. These 47 ligases were ultimately narrowed to select six variants (18, 24, 12, 8, 21, and 22) for further analysis.

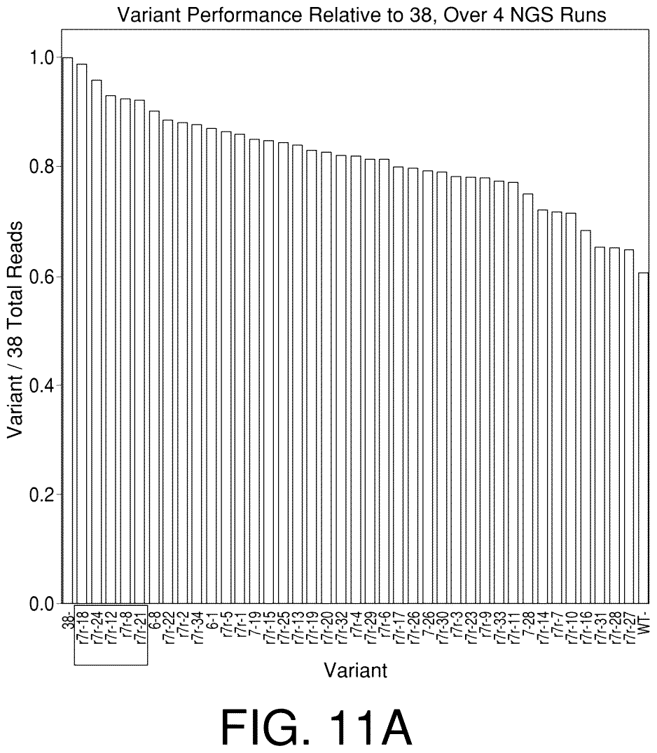

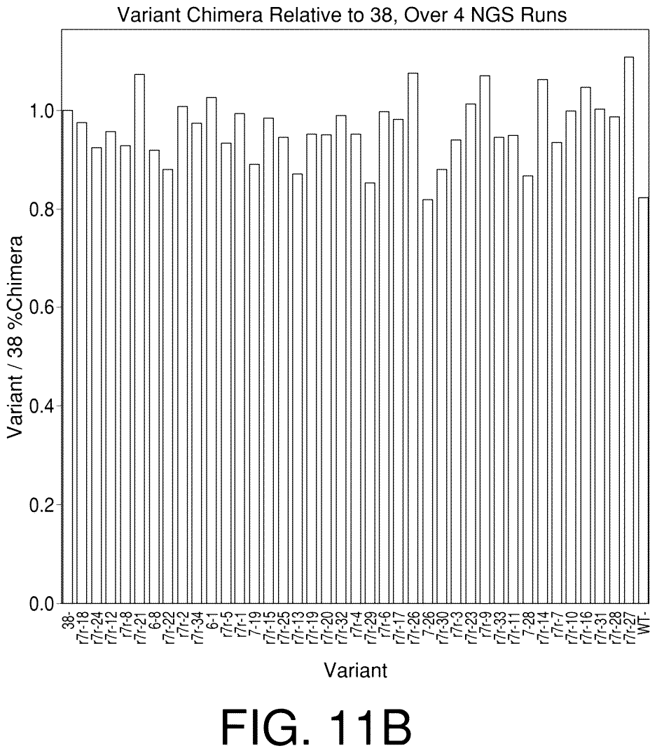

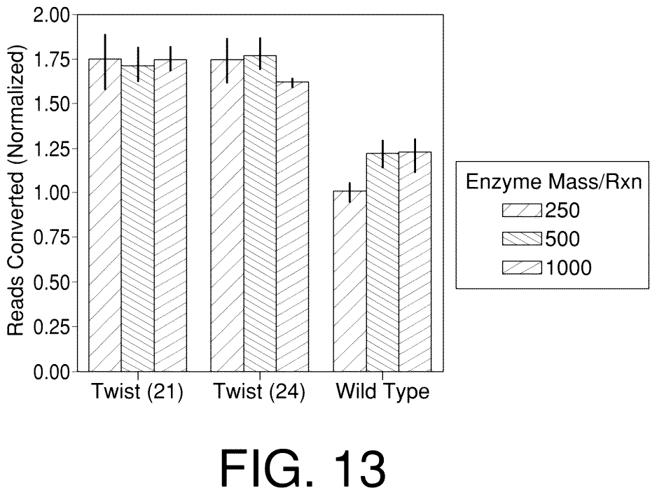

Results are shown in FIGS. 11A-13. All variants had a similar amount of chimeras, within the range of 0.1-0.2x of the best variant. Mutations in these variants are shown in Table 4.

TABLE 4

| Variant | Mutations (numbering relative to SEQ ID NO.: 1) |
|---|---|
| r7r-18 | E88K_V119R_Q223K_L293E_E440K_D448W |
| r7r-24 | E88K_V119R_Q223K_L293E_V372I_E483K |
| r7r-12 | E88K_V119R_Q223K_L293E_E440K |
| r7r-8 | E88K_T91M_V119R_G128K_Q223K_L293E_E440K |
| r7r-21 | E88K_V119R_G128K_E168K_Q223K_L231A_L293E_E440K |
| r7r-22 | E88K_V119R_Q223K_L293E_E440K_D448P |

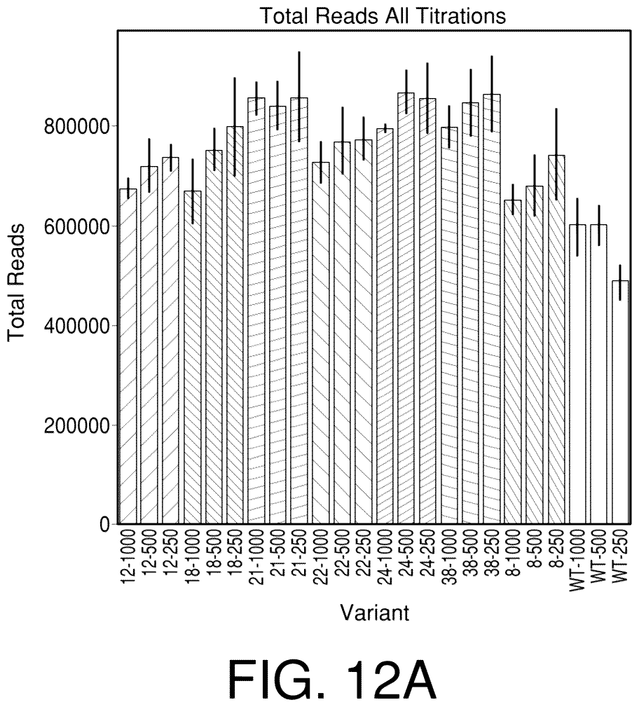

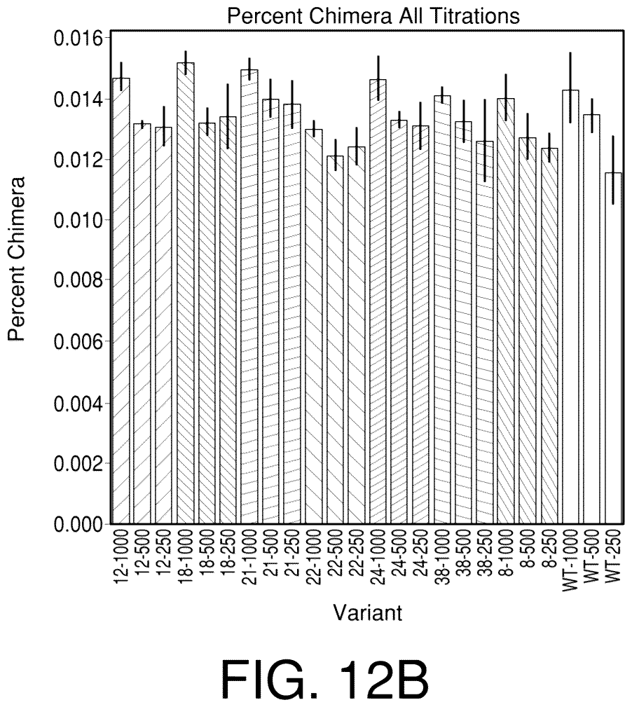

These six variants were then subjected to an additional NGS screen which varied the amount of enzyme (250, 500, and 1000 ng conditions). Two control reactions were included, and each condition was carried in four replicates. Enzyme variants generally performed better at lower mass loadings (FIGS. 12A-12B). Data against a wild type control is shown for variants 21 and 24. Variants r7r-21 and r7r-24 comprised protein SEQ ID NOS.: 2 and 3, and were expressed from nucleic acid SEQ ID NOS.: 5 and 6, respectively.

The present disclosure is further described by the following non-limiting items.

Item 1. A variant polypeptide comprising at least one amino acid mutation relative to SEQ ID NO.: 1.

Item 2. The polypeptide of item 1, wherein the polypeptide comprises at least 80% similarity to any one of SEQ ID NOS: 2-3.

Item 3. The polypeptide of item 1, wherein the polypeptide comprises at least 90% similarity to any one of SEQ ID NOS: 2-3.

Item 4. The polypeptide of item 1, wherein the polypeptide comprises at least 95% similarity to any one of SEQ ID NOS: 2-3.

Item 5. The polypeptide of item 1, wherein the polypeptide comprises at least 98% similarity to any one of SEQ ID NOS: 2-3.

Item 6. The polypeptide of item 1, wherein the polypeptide comprises any one of SEQ ID NOS: 2-3.

Item 7. The polypeptide of item 1, wherein the polypeptide comprises at least 10 contiguous amino acids of any one of SEQ ID NOS: 2-3.

Item 8. The polypeptide of item 1, wherein the polypeptide comprises at least 20 contiguous amino acids of any one of SEQ ID NOS: 2-3.

Item 9. The polypeptide of item 1, wherein the polypeptide comprises 20-100 contiguous amino acids of any one of SEQ ID NOS: 2-3.

Item 10. The polypeptide of item 1, wherein the polypeptide comprises at least 2 amino acid mutations relative to SEQ ID NO: 1.

Item 11. The polypeptide of item 1, wherein the polypeptide comprises at least 4 amino acid mutations relative to SEQ ID NO: 1.

Item 12. The polypeptide of item 1, wherein the polypeptide comprises at least 6 amino acid mutations relative to SEQ ID NO: 1.

Item 13. The polypeptide of any one of items 1-12, wherein the mutations are at one or more of positions E88, T91, V119, G128, E168, Q223, L231, L293, V372, E440, D448, and E483 relative to SEQ ID NO.: 1.

Item 14. The polypeptide of item 13, wherein the mutations are at one or more of positions E88, V119, G128, E168, Q223, L231, L293, and E440 relative to SEQ ID NO.: 1.

Item 15. The polypeptide of item 13, wherein mutations are at one or more of positions E88, V119, Q223, L293, V372, and E483 relative to SEQ ID NO.: 1.

Item 16. The polypeptide of item 13, wherein the mutations are selected from one or more of E88K, T91M, V119R, G128K, E168K, Q223K, L231A, L293E, V372I, E440K, D448W, D448P, and E483K relative to SEQ ID NO.: 1.

Item 17. The polypeptide of item 13, wherein the mutations are selected from one or more of E88K, V119R, G128K, E168K, Q223K, L231A, L293E, and E440K relative to SEQ ID NO.: 1.

Item 18. The polypeptide of item 13, wherein the mutations are selected from one or more of E88K, V119R, Q223K, L293E, V372I, and E483K relative to SEQ ID NO.: 1.

Item 19. The polypeptide of any one of items 1-18, wherein the polypeptide further comprises a purification tag.

Item 20. A nucleic acid encoding for the polypeptide of any one of items 1-19.

Item 21. A nucleic acid comprising at least 80% similarity to any one of SEQ ID NOS: 4-5, with the proviso the polypeptide does encode for a polypeptide of SEQ ID NO.: 1.

Item 22. The nucleic acid of item 21, wherein the nucleic acid of comprises at least 90% similarity to any one of SEQ ID NOS: 4-5.

Item 23. The nucleic acid of item 21, wherein the nucleic acid of comprises at least 95% similarity to any one of SEQ ID NOS: 4-5.

Item 24. A vector comprising the nucleic acid of any one of items 20-23.

Item 25. The vector of item 24, wherein the vector comprises a plasmid.

Item 26. A cell comprising the nucleic acid of any one of items 20-23.

Item 27. The cell of item 26, wherein the cell comprises a bacterial cell.

Item 28. A method of expressing the polypeptide of any one of items 1-19.

Item 29. The method of item 25, wherein expression comprises translation of the nucleic acid sequence of any one of items 20-23.

Item 30. The method of item 28 or 29, wherein the method comprises an in-vivo method.

Item 31. The method of item 28 or 29, wherein the method comprises a cell-free method.

Item 32. A method for forming a covalent bond between two nucleotides comprising contacting a first nucleotide and a second nucleotide with a polypeptide of any one of items 1-19.

Item 33. The method of item 32, wherein the first nucleotide and the second nucleotide are present on the same nucleic acid.

Item 34. The method of item 32, wherein the covalent bond forms a circular nucleic acid.

Item 35. The method of item 32, wherein the first nucleotide is present on a first nucleic acid and the second nucleotide is present on a second nucleic acid.

Item 36. The method of item 32, wherein the first nucleic acid and/or the second nucleic comprises genomic DNA or a fragment thereof.

Item 37. The method of item 32, wherein the first nucleic acid and/or the second nucleic comprises cDNA.

Item 38. The method of item 32, wherein the first nucleic acid and/or the second nucleic comprises an adapter.

Item 39. The method of item 38, wherein the first nucleic acid comprises a first adapter and genomic DNA or cDNA.

Item 40. The method of item 39, wherein the second nucleic acid comprises a second adapter.

Item 41. The method of any one of items 38-40, wherein the adapter comprises at least one barcode.

Item 42. The method of item 39, wherein the barcode comprises one or more of a sample index, a plate index, a cell index, and a unique molecular identifier.

Item 43. A method for preparing a nucleic acid library comprising
(a) providing one or more sample nucleic acids;
(b) contacting the one or more sample nucleic acids with a plurality of adapters and a polypeptide of any one of items 1-19 to form a nucleic acid sequencing library comprising adapter-ligated nucleic acids; and
(c) sequencing the nucleic acid library. The method of item 43, wherein the sample nucleic acids comprise genomic fragments.

Item 45. The method of item 43, wherein the genomic fragments are obtained from cleavage or amplification of a genome.

Item 46. The method of item 43, wherein the sample nucleic acids comprise cDNAs.

Item 47. The method of item 43, wherein the sample nucleic acids comprise cfDNAs.

Item 48. The method of any one of items 43-47, wherein the method further comprises one or more steps of end-repair, a-tailing, and amplification.

Item 49. The method of item 43-48, wherein the method further comprises enriching the nucleic acid library prior to sequencing.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A variant polypeptide having an amino acid sequence comprising at least one amino acid mutation relative to SEQ ID NO: 1.

2. The polypeptide of claim 1, wherein the polypeptide has an amino acid sequence comprising at least 80% similarity to SEQ ID NO: 2 or SEQ ID NO: 3.

3. The polypeptide of claim 1, wherein the polypeptide has an amino acid sequence comprising at least 90% similarity to SEQ ID NO: 2 or SEQ ID NO: 3.

4. The polypeptide of claim 1, wherein the polypeptide has an amino acid sequence comprising at least 95% similarity to SEQ ID NO: 2 or SEQ ID NO: 3.

5. The polypeptide of claim 1, wherein the polypeptide has an amino acid sequence comprising at least 98% similarity to SEQ ID NO: 2 or SEQ ID NO: 3.

6. The polypeptide of claim 1, wherein the polypeptide has an amino acid sequence comprising SEQ ID NO: 2 or SEQ ID NO: 3.

7. The polypeptide of claim 1, wherein the polypeptide has an amino acid sequence comprising at least 10 contiguous amino acids of SEQ ID NO: 2 or SEQ ID NO: 3.

8. The polypeptide of claim 1, wherein the polypeptide has an amino acid sequence comprising at least 20 contiguous amino acids of SEQ ID NO: 2 or SEQ ID NO: 3.

9. The polypeptide of claim 1, wherein the polypeptide has an amino acid sequence comprising 20-100 contiguous amino acids of SEQ ID NO: 2 or SEQ ID NO: 3.

10. The polypeptide of claim 1, wherein the polypeptide has an amino acid sequence comprising at least 2 amino acid mutations relative to SEQ ID NO: 1.

11. The polypeptide of claim 1, wherein the polypeptide has an amino acid sequence comprising at least 4 amino acid mutations relative to SEQ ID NO: 1.

12. The polypeptide of claim 1, wherein the polypeptide has an amino acid sequence comprising at least 6 amino acid mutations relative to SEQ ID NO: 1.

13. The polypeptide of claim 1, wherein the at least one amino acid mutation is at one or more of positions E88, T91, V119, G128, E168, Q223, L231, L293, V372, E440, D448, and E483 relative to SEQ ID NO: 1.

14. The polypeptide of claim 13, wherein the at least one amino acid mutation is at one or more of positions E88, V119, G128, E168, Q223, L231, L293, and E440 relative to SEQ ID NO: 1.

15. The polypeptide of claim 13, wherein the at least one amino acid mutation is at one or more of positions E88, V119, Q223, L293, V372, and E483 relative to SEQ ID NO: 1.

16. The polypeptide of claim 13, wherein the at least one amino acid mutation is selected from one or more of E88K, T91M, V119R, G128K, E168K, Q223K, L231A, L293E, V372I, E440K, D448W, D448P, and E483K relative to SEQ ID NO: 1.

17. The polypeptide of claim 13, wherein the at least one amino acid mutation is selected from one or more of E88K, V119R, G128K, E168K, Q223K, L231A, L293E, and E440K relative to SEQ ID NO: 1.

18. The polypeptide of claim 13, wherein the at least one amino acid mutation is selected from one or more of E88K, V119R, Q223K, L293E, V372I, and E483K relative to SEQ ID NO: 1.

19. The polypeptide of claim 1, wherein the polypeptide further comprises a purification tag.

20. A nucleic acid encoding for the polypeptide of claim 1.
